(19) United States
(12) Plant Patent Application Publication
(10) Pub. No.: US 2002/0129414 P1
(43) Pub. Date: Sep. 12, 2002

(54) CHRYSANTHEMUM PLANT NAMED 'ASTRO TIME'

(76) Inventor: Mark Roland Boeder, West Wittering (GB)

Correspondence Address: Mark K. Dickson, HOWREY SIMON ARNOLD & WHITE, LLP, 750 Bering Drive, Houston, TX, 77057-2198, US

(21) Appl. No.: 09/802,055

(22) Filed: Mar. 7, 2001

(51) Int. Cl. A01H 5/00

(52) U.S. Cl. PLT/288

(57) ABSTRACT

A new variety of Chrysanthemum plant named ‘Astro Time,’ having several rows of cream green, tubular rayflorets.

### BACKGROUND OF THE INVENTION

The present invention comprises a new and distinct cultivar of Chrysanthemum botanically known as Dendranthema grandiflora, and referred to by the cultivar name ‘Astro Time’.

‘Astro Time’, identified as AST1 originated from a cross made by Mark Boeder in a constrolled breeding program at Chichester, West Sussex, United Kingdom. The female parent of the new variety is an undistributed, unpatented seedling identified as 95283 and the male parent is an undistributed, unpatented seedling identified as 95223. The new variety ‘Astro Time’ has been asexually reproduced by vegetative cuttings at Chichester, West Sussex, United Kingdom and the distinguishing characteristics are retained through successive generations of asexual reproduction.

### BRIEF SUMMARY OF THE INVENTION

‘Astro Time’ is a pot type of Chrysanthemum plant variety having quilled, decorative-type flowers and a cream green flower color.

Plants of the new Chrysanthemum variety ‘Astro Time’ are similar to the female parent ‘95283’ in flower type. In side-by-side comparisons at Chichester, West Sussex, United Kingdom, under commercial practice, plants of the new Chrysanthemum variety ‘Astro Time’ differed from plants of the female parent ‘95283’ in the following characteristics.

1. The new variety ‘Astro Time’ produces cream green flowers whereas the female parent ‘95283’ produces white flowers.

2. Plants of the new variety ‘Astro Time’ have larger flowers than plants of the female parent ‘95283’.

### Comparison with Other Varieties

Plants of the new Chrysanthemum variety ‘Astro Time’ are similar to the unpatented cultivar ‘Ice Time’ in plant habitat and growth rate. However, in side-by-side comparisons at Chichester, West Sussex, United Kingdom, under commercial practice, plants of the new Chrysanthemum variety ‘Astro Time’ differed from plants of the cultivar ‘Ice Time’ in the following characteristics.

1. The new variety ‘Astro Time’ produces cream green flowers whereas the cultivar ‘Ice Time’ produces pure white flowers.

2. Plants of the new variety ‘Astro Time’ have quilled petals and plants of the cultivar ‘Ice Time’ have spoon shaped rayflorets.

### BRIEF DESCRIPTION OF ILLUSTRATIONS

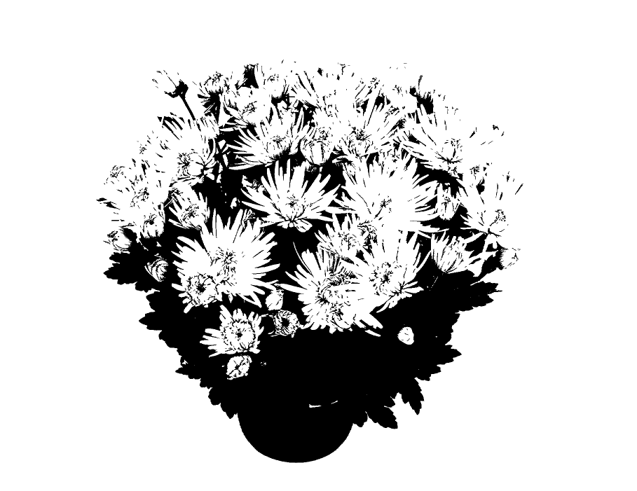

Typical specimens of the plant and flowers for the new Chrysanthemum variety ‘Astro Time’ are shown in the accompanying photograph. The colors shown are as true as possible within the usual limits of this kind of illustration. FIGURE 1 is a whole plant view of the new variety ‘Astro Time’ grown in a pot.

### DETAILED BOTANICAL DESCRIPTION

The following description of the new Chrysanthemum variety ‘Astro Time’ is of plants grown in a greenhouse at Chichester, West Sussex, United Kingdom in the month of December. The cultivar has not been observed under all possible environmental conditions. The phenotype may vary significantly with variations in the environment such as temperature, length of day and light intensity, without any variance in genotype. The commercial classification of the new variety is a pot Chrysanthemum.

Plants of the new variety have been grown successfully under temperature conditions averaging about 18° C. at night and about 18° C. to 24° C. during the day under light conditions of about 5,000 to 6,000 foot candles. The plants respond well to the use of growth retardant, such as one B9 treatment at about 1 gram/liter. To produce a commercial product the plants may be pinched once. The typical container size for commercial growth is 1 liter. It has been observed that the shelf life of the new variety is about 20 days with a response time of about 7.5 weeks.

The new variety may be produced as a spray or disbudded pot chrysanthemum. The fo Llowing description is with respect to a plant produced as spray pot Chrysanthemum. In the description of this new Chrysanthemum variety, color values have been taken from The Royal Horticultural Society Colour Chart (R.H.S.C.C.).

### Plant

Plant type: pot Chrysanthemum

Habit: upright

Height: 20-25 cm

Branching characteristics (describe): upright spreading

Length of Lateral Branches: 15-20 cm

Number of Breaks from Pinch: 3-5

Stem Color: near 139C

Response Time: 7.5 weeks

Vigor: short

Shelf Life: 18 days

Disease (susceptibility or resistance observed): no observations Growth retardant type and treatment: 1 application B9 (1 gram/liter)

Propagation:

Type.—vegetative propagation via stem cuttings

Time to rooting.—14 days with soil temperatures of 18° C.

Rooting habit.—After 7 Long Days (20 hrs light) first roots emerge from root primordia. After 14 Long Days a complete root system is developed.

Foliage Number of Leaves Per Lateral Branch: 10-16 Compound or single: single Arrangement of Leaves: alternate

Shape of Leaf.—typical 5 lobed, oblong.

Size of Leaf.—Width (cm): 2.0-4.5 Length (cm): 4.0-8.0 cm.

Leaf Apex.—mucronate.

Base.—cordate.

Attachment.—petioled.

Aspect.—slight undulating.

Margin.—palmately lobed.

Surface characteristics: Top: pubescent Bottom: pubescent

Petiole:

Color.—near 139A.

Length.—1.0-2.0 cm Venation (describe): prominent midvein.

Color.—upper side: near 138B under side: near 138C.

Color:

Mature Leaf, upper side.—near 139A; under side: near 137B.

Young Leaf, upper side.—near 137A; under side: near 137C.

### Flower

Flower appearance: many rows of quilled ray florets and several rows of tubular florets

Flower type: semi-decorative

Number of blossoms per branch: 3-5

Inflorescence form.—cyme

Depth of fully expanded blossoms.—2.0-3.0 cm.

Diameter of fully expanded blossoms.—7.0-9.0 cm.

Sepals:

Number.—20-25 .

Color.—near 139B.

Length.—0.5-0.8 cm.

Width.—0.1-0.4 cm.

Texture/Appearance.—pubescent.

Peduncle: more or less even in length: longest length is on average 3.0-4.0 cm, shortest length is on average 1.0-3.0 cm

Length.—1.0-4.0 cm.

Color.—near 138B.

Surface.—pubescent.

Habit.—slight undulating.

Strength.—medium.

Pedicel:

Length.—terminal: 13.0-14.0 cm; lateral 11.0-13.0 cm.

Color.—near 138B.

Surface.—pubescent.

Habit: slight undulating.—

Strength.—medium.

Ray Florets:

Form/Shape.—quilled.

Texture/Appearance.—matte.

Number per flower.—250-300 .

Length.—0.6-3.9 cm Width: 0.1-0.4 cm.

Apex.—dentated.

Base.—tubular.

Margin.—entire.

Disc Florets:

Form/Shape.—tubular.

Texture/Appearance.—matte.

Number per flower.—0-50 .

Length.—0.1-0.4 cm; Width 0.05-0.1 cm.

Diameter of disc: 0.0-0.4 cm.

Fragrance: faint

Flower bud (at onset of color).—

Length.—0.5-0.7 cm

Diameter.—0.7-1.0 cm

Form/shape.—spherical

General Flower Color:

1. Ray florets, upper side.—Immature: near145C Mature: near 157A Older/Fading: near 157C.

2. Ray florets, under side.—Immature: near 145C Mature: near 157A Older/Fading: near 157C.

3. Disc florets.—Immature: near 138A Mature: near 13B Older/Fading: near 13C.

4. Bud.—near 150A.

Flower progression with age: Flower color fades from green via white-green to white with age.

### Reproductive Organs

Ray Florets: Pistillate

Pistil Number.—about 5.

Stigma color.—near 6B

Stigma shape.—fork

Style Color.—near 142C.

Style Length.—0.05-0.3 cm

Disc Florets: Pistillate

Pistil Number.—about 5

Stigma color.—near 6B

Stigma shape.—fork

Style Color.—near 142C

Style Length.—0.05-0.2 cm

What is claimed is:

1. A new and distinct variety of Chrysanthemum plant, substantially as described and illustrated herein.
